(19) United States
(12) Patent Application Publication
(10) Pub. No.: US 2003/0086816 A1
(43) Pub. Date: May 8, 2003

(54) Process for removing iron oxide deposit from a metal surface in contact with an aqueous system

(76) Inventors: Douglas B. Dewitt-Dick, Portland, TX (US); Kaj D. Rondum, Budd Lake, NJ (US)

Correspondence Address: David L. Hedden, Ashland Inc., P.O. Box 2219, Columbus, OH, 43216, US

(21) Appl. No.: 10/176,090

(22) Filed: Jun. 20, 2002

Related U.S. Application Data
(63) Continuation-in-part of application No. 09/287,709, filed on Apr. 7, 1999, now abandoned.
(60) Provisional application No. 60/081,259, filed on Apr. 9, 1998.

(51) Int. Cl. C23F 11/00; C23F 11/10; A61L 9/00

(52) U.S. Cl. 422/7; 422/17; 422/28; 422/32

(57) ABSTRACT

This invention relates to a process for removing iron oxide deposit from a metal surface in contact with an aqueous system, in particular the metal surface of a boiler operating at high pressures. The process comprises adding an elevated dosage of an oxime to the aqueous system in an amount effective to partially or entirely remove iron oxide deposits from the metal surface.

### CROSS REFERENCE TO RELATED APPLICATIONS

This application is a continuation-in-part of application Ser. No. 09/287,709, filed on Apr. 7, 1999, which is a continuation-in-part of provisional application serial No. 60/081,259, filed on Apr. 9, 1998, both of which are hereby incorporated by reference.

### CLAIM TO PRIORITY

Applicants claim the priority date of application Ser. No. 09/287,709, filed on Apr. 7, 1999, and provisional application serial No. 60/081,259, filed on Apr. 9, 1998.

### FIELD OF THE INVENTION

This invention relates to a process for removing iron oxide deposit from a metal surface in contact with an aqueous system, in particular the metal surface of a boiler operating at high pressures. The process comprises adding an elevated dosage of an oxime to the aqueous system in an amount effective to partially or entirely remove iron oxide deposits from the metal surface.

### BACKGROUND OF THE INVENTION

The mitigation of corrosion in steam generating systems is vital to the continued efficient operation of the systems. Oxygen pitting can rapidly lead to failures while formation of metal oxides results in deposition, causing reduced heat transfer rates and under-deposit corrosion. The limited deposit tolerances in high-pressure boilers require that the corrosion inhibition program perform optimally.

The application of methyl ethyl ketoxime (MEKO) as an effective oxygen scavenger and metal passivator is well established and thoroughly documented in the industry. See, for instance, U.S. Pat. No. 4,487,745. Although this patent indicates that the amount of oxime used in treating boiler water is from 0.0001 ppm to 500 ppm, more usually 1.0 ppm to 50 ppm¹, commercial experience with MEKO indicates that the typical dosage of MEKO used to control feedwater oxygen scavenging is from 30-80 ppb in boilers operating at high pressures, i.e. boilers that operate at pressures of at least 1,000 psi.

MEKO controls corrosion in the feedwater circuit by scavenging oxygen and by establishing a corrosion-resistant oxide film on waterside metallic surfaces. Since MEKO is volatile, these effects are also extended to post-boiler steam and condensate areas. Since many, if not most systems employ a means for returning condensed steam to supplement boiler feedwater requirements, effective corrosion inhibition can reduce the amount of iron oxide corrosion products that return to the boiler. This is of particular concern in systems that do not use condensate polishers for iron removal. Over time, these iron oxide deposits accumulate on heat transfer surfaces within the boiler affecting thermal efficiency and compromising boiler tube metal integrity.

In addition to oxygen scavenging and the prevention of iron oxide corrosion, there is an interest in removing deposits from metal surfaces in operating boilers in order to reduce cleaning frequency and promote safer operating conditions. In fact, a number of professional organizations including ASME and EPRI have published suggested feedwater iron and boiler deposit weight density (DWD) guidelines. DWD is defined as the weight of deposit removed from the waterside surface of a boiler tube, divided by the surface area from which it was removed.² The units of the DWD value can be expressed in milligrams per square centimeter or grams per square foot³. ¹ See column 3, lines 24-33 of the patent. ² Deposit removed from the hot or furnace side of the tube is measured using the mechanical scraping method (ASTM D3483 Method A). ³The metric and English units are approximately equivalent in this case.

Although no uniform policy exists related to how DWD values correlate to boiler cleanliness, boiler manufacturers and others have established guidelines to assist boiler operators in determining when to clean a boiler. In addition, boiler inspectors, insurance companies, and boiler manufacturers have established DWD limits to schedule the cleaning of boilers in order to mitigate stress and failures, and promote equipment longevity and life

expectancy. A summary of some select guidelines for when to clean boilers is shown in Table I below.

TABLE I
GUIDELINES FOR CLEANING OF BOILERS

| Unit Operating Pressure (psig) | DWD (gm/ft²)/Suggested Cleaning |
|---|---|
| <1000 | 20-40 |
| 1000-2000⁴ | 12-20 |
| >2000 | 10-12 |

⁴Including all Kraft Recovery and Refuse-Fired Boilers.

Table I shows that the importance of deposit control takes on greater significance for boilers operating at higher pressure boilers, i.e. boilers operating at pressures of at least 1,000 psig. This is due to the critical nature of these operating units and their limited tolerance to deposition particularly on the hot side of waterside boiler tubes in the generating section. In many plants, condensate polishers are used to help reduce deposits from accumulating or more frequent cleaning must take place in order to remove deposits.

In view of this potential for iron oxide accumulation in boilers operating at higher pressures, other authorities suggest limits on the concentration of iron (typically as a result of corrosion) in the boiler feedwater which leads to accumulation of iron oxide deposits in a boiler. For instance, see Table II below which is reprinted with the permission of ASME from “Consensus on Operating Practices for the Control of Feedwater and Boiler Water Chemistry in Modem Industrial Boilers”.

TABLE II
SUGGESTED FEEDWATER CHEMISTRY LIMITS
(INDUSTRIAL WATERTUBE, HIGH DUTY, PRIMARY FUEL FIRED, DRUM
TYPE)

MAKEUP WATER PERCENTAGE: Up to 100% of feedwater.
CONDITIONS: Includes superheater, turbine drives, or process restriction on steam
purity.
LIMIT OF TOTAL IRON (Fe) AT VARIOUS OPERATING PRESSURES (see below):

| | | | | | |
|---|---|---|---|---|---|
| DOP⁵ (psig) | 601-750 | 751-900 | 901-1000 | 1001-1500 | 1501-2000 |
| Fe⁶ ppm (mg/l) | ≦0.025 | ≦0.02 | ≦0.02 | ≦0.01 | ≦0.01 |
| Fe⁷ (pbb) | ≦25 ppb | ≦20 ppb | ≦20 ppb | ≦10 ppb | ≦10 ppb |

⁵ Drum Operating Pressure. ⁶ Total iron ppm. ⁷ Total iron ppb.

Table II indicates that less total iron can be tolerated as the direct operating pressure increases. In view of this, it is clearly useful to find ways of reducing or eliminating iron oxide deposit on the metal surfaces of a boiler at higher pressure, i.e. pressures of at least 1,000 psig.

Although MEKO is an effective oxygen scavenger and operates to control the formation of iron oxide corrosion on metal surfaces, the prior art does not disclose the use of MEKO to remove iron oxide deposits from metals in contact with an aqueous system.

### SUMMARY OF THE INVENTION

This invention relates to a process for removing iron oxide deposits from a metallic surface of a boiler operating at a presssure of a least 1,000 psig, wherein the metallic surface is in contact with an aqueous system. The process comprises adding at least 300 ppb of an oxime to the water circulating through the boiler operating at high pressure.

The addition of an oxime to an aqueous system reduces the existing iron oxide deposit accumulated on the metal surface in contact with the aqueous system. This reduction is shown by a reduction in the deposit weight density (DWD). This result is achieved by adding sufficient oxime to partially or totally remove iron oxide deposit on

metals in contact with an aqueous system, and maintaining this oxime dosage for an appropriate time.

The use of this process results in cost savings because there is less need for frequent cleanings of the operating equipment, i.e. boiler, if the process is used. Further savings result by using this process because heat generated by the boiler is more efficient since there is lower deposit thickness and density on the metal of the boiler tubes. The corrosion potential for the boiler tube surfaces is also greatly reduced when this process is used.

### BEST MODE AND OTHER MODES

The oximes used in this process are described in U.S. Pat. Nos. 4,487,745 which is hereby incorporated by reference and shown by the following chemical structure:

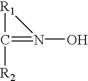

wherein R₁ and R₂ are the same or different and are selected from hydrogen, lower alkyl groups of 1-8 carbon atoms and aryl groups, and mixtures thereof, particularly aliphatic oximes. Most preferably used as the oxime is methyl ethyl ketoxime (MEKO). The oxime is preferably added to a feedpoint that will expose the said methyl ethyl ketoxime to a temperature of about 30° C. to about 320° C.

As was mentioned previously, the typical dosage of oxime, used in boilers operating at higher pressures for feedwater oxygen scavenging, is in the range of 30-80 ppb. The unique aspect of the subject process is that the effective dosage of the oxime in the aqueous system required for deposit weight density (DWI) reduction is about 10-20 times the amount used for oxygen scavenging. In other words, the dosage of oxime needed to reduce iron deposits in boiler is at least 300 ppb, preferably at least 500 ppb, and most preferably, at least 600 ppb. However, the oxime dosage, in most cases, would not be expected to exceed 1000 ppb. Typically, the oxime dosage is maintained in an operating system for a minimum of one week, preferably from two to eight weeks. Typically, the increased oxime dosage would not be expected to exceed 12 weeks.

Although it may be possible to use the subject process for treating other aqueous systems, it is particularly useful for treating boiler water because the pressures reached in a boiler are such that iron oxide deposit formation becomes less tolerable.

### EXAMPLE

The power plant, where the increased dosage of MEKO was fed, was a 165 megawatt steam turbine driven generator connected to a high pressure, 1870 psig gas-fired natural circulation Babcock and Wilcox boiler. The unit contained a deaerating feedwater heater, but did not contain condensate polishers for iron oxide removal. The boiler operated without the use of MEKO prior the introduction of MEKO at elevated dosages.

MEKO was added to the system feedwater at the condenser hotwell. The dosage of MEKO in the feedwater was about 600 ppb and was maintained at this dosage for period of about three to four weeks. Later, the boiler was removed from service, inspected, and two waterwall tube samples (MEKO-treated sample tubes) were taken and analyzed for DWD (deposit weight density).

DWD⁸ was measured according to ASTM D3483 on the hot side of the two MEKO-treated sample tubes as well as on two previously removed similar sample tubes taken from the same area in the generating section of the boiler prior to the time MEKO was used in the boiler. The results are shown Table III below. ⁸ As was mentioned before, the DWD (deposit weight density) measurement is defined as the weight of deposit removed from the waterside surface of a boiler tube, divided by the surface area from which it was removed.

TABLE III
COMPARISON OF IRON OXIDE DEPOSIT BEFORE AND AFTER
USING MEKO
DWD (gm/ft²)

| | BEFORE MEKO | AFTER | % DEPOSIT |
|---|---|---|---|
| Example | (Comparison) | MEKO | REMOVAL |
| 1 | 101.7 | 8.9 | 92% |
| 2 | 22.1 | 5.2 | 78% |

The data in Table III show significant deposit removal, amounting to 92% and 78% respectively, when MEKO is added to the aqueous system on-line at elevated levels, i.e. at dosages about ten times its normal dosage. This degree of removal, or any removal for that matter, was not expected.

We claim:

1. A process for removing iron oxide deposits from a metallic surface of a boiler operating at a presssure of a least 1,000 psig, wherein said metallic surface is in contact with an aqueous system, comprising:
adding at least 300 ppb of an oxime to said aqueous system.

2. The process of claim 1 wherein the oxime is methyl ethyl ketoxime.

3. The process of claim 2 wherein the oxime is added to a feedpoint which will expose the said methyl ethyl ketoxime to a temperature between 30° C. to 320° C.

4. The process of claim 3 wherein the operating pressure of the boiler is at least 2,000 psig.

5. The process of claim 6 wherein the methyl ethyl ketoxime is added to the aqueous stream at a dosage of at least 600 ppb.

6. The process of claim 6 wherein dosage of methyl ethyl ketoxime is maintained for a period of at least four weeks.
